(19) United States
(12) Patent Application Publication
(10) Pub. No.: US 2023/0296603 A1
(43) Pub. Date: Sep. 21, 2023

(54) DEVELOPMENT OF A SMARTPHONE-BASED BIOSENSOR DEVICE FOR DETECTING SARS-COV-2 ANTIGENS IN BODY FLUIDS USING LOCALIZED SURFACE PLASMON RESONANCE (LSPR)

(71) Applicants: Minh Van PHAN, Bien Hoa City (VN); Board of Supervisors of Louisiana State University and Agricultural and Mechanical College, Baton Rouge, LA (US)

(72) Inventors: MINH VAN PHAN, Baton Rouge, LA (US); LUAN D. VU, Baton Rouge, LA (US); STEPHANIA A. CORMIER, Baton Rouge, LA (US); MANAS GARTIA, Baton Rouge, LA (US); ANH THI QUYNH PHAN, Baton Rouge, LA (US)

(21) Appl. No.: 18/006,560

(22) PCT Filed: Jul. 23, 2021
(86) PCT No.: PCT/US2021/042985

§ 371 Date: Jan. 23, 2023

Related U.S. Application Data
(60) Provisional application No. 63/056,102, filed on Jul. 24, 2020.

(51) Int. Cl. G01N 33/569; G01N 33/543; G01N 21/77

(52) U.S. Cl. CPC G01N 33/56983; G01N 33/54373; G01N 21/7703; G01N 2333/165; G01N 2469/10; B01L 2300/0654; B01L 2200/0652; G01N 2021/7716; G01N 2021/757

(57) ABSTRACT

The present disclosure presents nanostructure-based localized surface plasmon resonance systems and related methods. In this regard, a method comprises applying a body fluid sample to a metal surface of the nanostructure-based LSPR biosensor with linker, intermediate, and capture/probe antibodies; illuminating the metal surface of the nanostructure-based LSPR biosensor with the monochromatic, broadband, or laser light; measuring an intensity or spectrum of absorbed, reflected, transmitted, or scattered exiting light from the nanostructure-based LSPR biosensor having the body fluid sample and comparing the measured intensity or spectrum with a reference intensity; detecting a spectral shift of exiting light from the nanostructure-based LSPR biosensor having the body fluid sample; and signaling that the body fluid sample is positive for a presence of a particular biomaterial in response to detecting the spectral shift of the exiting light, wherein the biomaterial has binded or adsorbed to the metal surface of the nanostructure-based LSPR biosensor.

### CROSS-REFERENCE TO RELATED APPLICATION

This application claims priority to U.S. provisional application entitled, “Development of a Smartphone-Based Biosensor Device for Detecting SARS-COV-2 Antigens in Body Fluids Using Localized Surface Plasmon Resonance (LSPR),” having Ser. No. 63/056,102, filed Jul. 24, 2020, which is entirely incorporated herein by reference.

### TECHNICAL FIELD

The present disclosure is generally related to biosensors and related methods.

### BACKGROUND

Severe Acute Respiratory Syndrome Coronavirus-2 (SARS-CoV-2) is found to be the causative agent of the COVID-19 pandemic. As of May 13, 2020, there were 4,170,424 confirmed cases of COVID-19, with 287,399 deaths globally per WHO (World Health Organization) situation report-114. Although extensive quarantine and control methods were implemented by federal governments, the spread of the infection continued to be severe. SARS-CoV-2 has posed a life-threatening situation to the world population, disrupted normal life, and paralyzed the world economy. The widespread availability of accurate and rapid testing procedures is critical in helping identify COVID-19 carriers and control the spreading of the highly contagious and highly pathogenic SARS-CoV-2.

Currently, the identification of individuals who are actively infected with SARS-CoV-2 mainly relies on real-time reverse transcription polymerase chain reaction (qRT-PCR) amplification of the viral genetic material collected in nasopharyngeal and oropharyngeal swabs. Although showing high sensitivity and specificity, these assays required a high workload and can only be performed by well-trained laboratory personals in biosafety level 3 laboratories. Therefore, these assays are mainly performed on patients displaying COVID-19-related symptoms. However, a significant fraction of infected individuals remain asymptomatic. These contagious and asymptomatic individuals often go undetected and contribute to the spread of the disease.

### SUMMARY

Embodiments of the present disclosure provide nanostructure-based localized surface plasmon resonance systems and related methods. Briefly described, one embodiment of the system, among others, can comprise a nanostructure-based LSPR (localized surface plasmon resonance) biosensor having a linker molecule or polymer chain or nucleic acid (DNA, RNA) or peptide strand or capture antibody or polydopamine or alkane thiol or synthetic molecules with varying carbon chains comprised of —NH₂ or —COOH or —SH group at one end or both ends of the molecules to the nanostructure-based LSPR biosensor; an intermediate layer comprised of polyethylene glycol (PEG) or streptavidin or avidin or biotin or Polyethylenimine (PEI) or polyaziridine or dextran to the linker molecule; and probe antibodies or primary antibodies and/or secondary antibodies applied to the intermediate layer, wherein opposing ends of the biosensor are secured with ceramic ferrules; and a flow cell having an internal chamber, a top portion, a bottom portion, and opposing end portions, wherein the nanostructure-based LSPR biosensor is insertable within the internal chamber and is held in place by engaging the ends of the biosensor within holes at each end of the internal chamber. Further, the holes at each end of the internal chamber are configured to pass incoming light from one end of the flow cell to the opposite end of the flow cell across a length of the nanostructure-based LSPR biosensor; and a top portion of the flow cell includes an inlet hole for inserting a body fluid sample comprised of saliva, blood, sweat, tear, or cerebrospinal fluid to the nanostructure-based LSPR biosensor.

In one or more aspects for such systems, the system can comprise a light source positioned at one end of the flow cell to provide the incoming light through the hole at the one end of the flow cell; and/or a camera positioned at an opposite end of the flow cell to capture an image of exiting light from hole at the opposite end of the flow cell. In one or more aspects for such systems, the nanostructure-based LSPR biosensor can comprise an AuNP-coated fiber optic LSPR biosensor. In various embodiments, the nanostructure-based LSPR biosensor comprises an Au (gold) or Ag (silver) or Al (aluminum) or Cu (copper) or alloy of one of these nanoparticles (NP) coated fiber optic LSPR biosensor, wherein a shape of the nanoparticles can be sphere, cube, triangle, star, or rod, wherein the NP can be coated with oxide materials made of SiO₂, TiO₂, MnO₂ or other metal oxides to make a core-shell structure. Accordingly, in various embodiments, the nanostructure-based LSPR biosensor can comprise an Au/Ag/Al/Cu/Fe/Mn, or their alloy, or their oxide NP-coated fiber optic LSPR biosensor.

Briefly described, one embodiment of the method, among others, comprises illuminating a metal surface of a nanostructure-based LSPR (localized surface plasmon resonance) biosensor directly or using an optical fiber with a monochromatic, broadband, or laser light; measuring a reference intensity or spectrum of absorbed, reflected, transmitted or scattered light from the nanostructure-based LSPR biosensor; applying a linker molecule or polymer chain or nucleic acid (DNA, RNA) or peptide strand or capture antibody or polydopamine or alkane thiol or synthetic molecules with varying carbon chains comprised of —NH₂ or —COOH or —SH group at one end or both ends of the molecules to the metal nanostructure LSPR biosensor; applying an intermediate layer comprised of polyethylene glycol (PEG) or streptavidin or avidin or biotin or Polyethylenimine (PEI) or polyaziridine or dextran to the linker molecule; applying probe antibodies or primary antibodies and/or secondary antibodies to the intermediate layer; applying a body fluid sample comprised of saliva, blood, sweat, tear, or cerebrospinal fluid to a metal surface of the nanostructure-based LSPR biosensor with linker, intermediate, and capture/probe antibodies; illuminating the metal surface of the nanostructure-based LSPR biosensor directly or using an optical fiber with the monochromatic, broadband, or laser light; measuring an intensity or spectrum of absorbed, reflected, transmitted, or scattered light from the nanostructure-based LSPR biosensor having the body fluid sample and comparing the measured intensity or spectrum with the reference intensity; detecting a spectral shift of the absorbed, reflected, transmitted, or scattered light from the nanostructure-based LSPR biosensor having the body fluid sample; and/or signaling that the body fluid sample is positive for a presence of a particular biomaterial in response to detecting the spectral shift of the absorbed, reflected, transmitted, or scattered light, wherein the biomaterial has binded or adsorbed to the metal surface of the nanostructure-based LSPR biosensor.

In one or more aspects for such systems/methods, the biomaterial comprises S, N, or M proteins of SARS-CoV-2; a light source for the light is a smartphone LED flash, standalone LED, laser, continuum laser light, pulsed laser light, or broadband source; an internal camera of the smartphone or charged coupled device (CCD) or photometer or photodiode is used in measuring the intensity or spectrum of the absorbed, reflected, transmitted or scattered light from the nanostructure-based LSPR biosensor; and/or the nanostructure-based LSPR biosensor comprise an AuNP-coated fiber optic LSPR biosensor. In various embodiments, the nanostructure-based LSPR biosensor comprises an Au (gold) or Ag (silver) or Al (aluminum) or Cu (copper) or alloy of one of these nanoparticles (NP) coated fiber optic LSPR biosensor, wherein a shape of the nanoparticles can be sphere, cube, triangle, star, or rod, wherein the NP can be coated with oxide materials made of SiO₂, TiO₂, MnO₂ or other metal oxides to make a core-shell structure.

In one or more aspects for such systems/methods, an exemplary system/method can further perform operations comprising applying an electric current to the nanostructure-based LSPR biosensor to disassociate any binding of the biomaterial with surface of the nanostructure-based LSPR biosensor, wherein the electric current is optionally supplied by the smartphone or external battery or power source; reusing the nanostructure-based LSPR biosensor to test for the presence of the biomaterial in a new body fluid sample; inserting the nanostructure-based LSPR biosensor in a chamber of a flow cell, wherein the body fluid sample is applied to the nanostructure-based LSPR biosensor by being injected into an inlet hole of the flow cell; wherein the metal surface of the nanostructure-based LSPR biosensor is illuminated by passing the monochromatic, broadband, or laser light into a hole at one end of the flow cell that is coupled to one end of the nanostructure-based LSPR biosensor; capturing an image of the monochromatic, broadband, or laser light exiting from a hole at an opposite end of the flow cell that is coupled to an opposite end of the nanostructure-based LSPR biosensor, wherein the intensity of the absorbed, reflected, transmitted or scattered light from the nanostructure-based LSPR biosensor having the body fluid sample is determined by computing an average intensity of the captured image; and/or wherein each end of the flow cell contains a plurality of holes for holding a plurality of nanostructure-based LSPR biosensors, the plurality of nanostructure-based LSPR biosensors including a biosensor for sensing SARS-CoV-2 N protein, a biosensor for sensing SARS-CoV-2 antibodies against SARS-CoV-2 S protein, a biosensor used as isotype controls for specificity, and a biosensor used as a reference control.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows the binding of biomolecules (such as antibodies and antigens) at a metal surface in accordance with a surface plasmon resonance (SPR) phenomenon and the detection of a change in reflected light using an optical detector.

FIG. 1B shows the binding of biomolecules (such as antibodies and antigens) at a metal surface in accordance with a localized surface plasmon resonance (LSPR) phenomenon and the detection of a change in energy intensity of the reflected light using a smartphone in accordance with various embodiments of the present disclosure.

FIG. 2 illustrates an exemplary smartphone-based biosensor system in accordance with embodiments of the present disclosure

FIGS. 3A and 3B illustrate varying views of a flow cell assembly of the biosensor system of FIG. 2 in accordance with embodiments of the present disclosure.

FIGS. 4A-4B show varying views of an exemplary optical tube and related components of the biosensor system of FIG. 2 in accordance with embodiments of the present disclosure.

FIG. 4C presents a schematic of an exemplary nanostructured-based fiber optic LSPR biosensor within a flow cell assembly in accordance with embodiments of the present disclosure.

FIG. 5A presents an exemplary flow diagram of an exemplary biosensor testing application in accordance with embodiments of the present disclosure.

FIG. 5B shows a display representation of pinholes of an exemplary flow cell by an exemplary biosensor testing application in accordance with embodiments of the present disclosure.

FIG. 6 shows a composite of images of prototype components of an exemplary nanostructured-based fiber optic LSPR biosensor and flow cell assembly in accordance with embodiments of the present disclosure.

FIG. 7 shows a captured image of exiting light from an exemplary flow cell assembly in accordance with embodiments of the present disclosure.

FIG. 8 provides a schematic of a computing device according to various embodiments of the present disclosure.

### DETAILED DESCRIPTION

The present disclosure presents various embodiments of a wearable device for detecting SARS-CoV-2 antigens in body fluids using a nanostructure-based localized surface plasmon resonance (LSRP) biosensor that can be interfaced with a mobile device (e.g., smartphone). Designedly, an exemplary self-integrated testing device can be used as either at-home or at-field diagnostic tools for COVID-19.

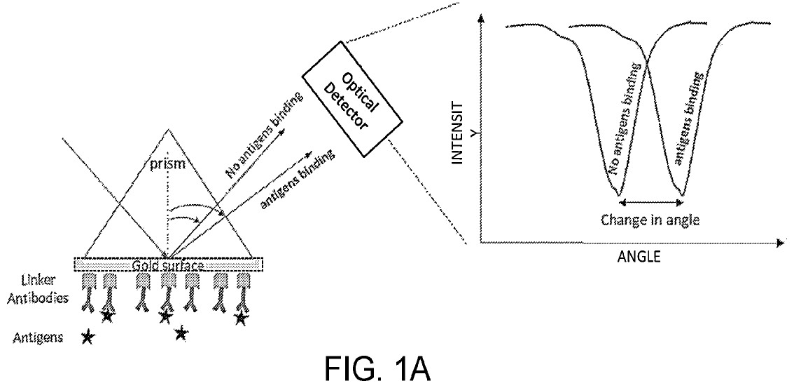

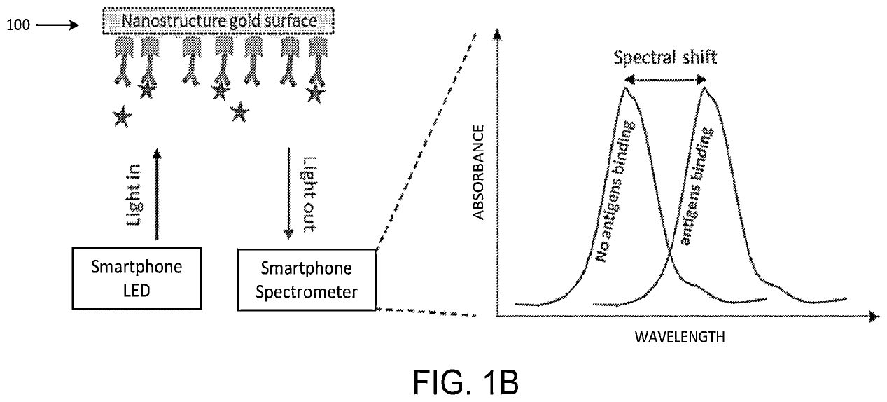

In brief, the last two decades have seen a tremendous development of surface plasmon resonance (SPR) use in biomedical applications. SPR is an optical phenomenon occurring when an incident light beam strikes a metal surface (typically silver or gold) at a particular angle. As illustrated in FIG. 1A, in the event of binding of biomolecules (such as antibodies and antigens) at the metal surface, SRP phenomenon results in changes of the angle of the reflected light. Since the changes in the angle (so-called reflection index) is measurable and is directly correlated to the thickness of the molecular layers at the metal surface, the binding of biomolecules (antibodies or antigens) to the interface is detected. However, SPR is instrumentally complex, and SPR is only achieved by using adaptive optics and thermal controls which pose a formidable obstacle for developing miniaturized testing devices.

In contrast, as demonstrated by FIG. 1B, an exemplary nanostructure-based LSPR (localized surface plasmon resonance) biosensor 100 is a label-free sensing technique relying on spectral shifts instead of a change in the angle of reflected light. Nanostructure-based LSPR occurs when an incident light beam (a monochromatic, broadband, or laser light) from a light source (e.g., LED flash or flashlight from a smartphone) strikes the nanostructure metal surface (typically silver or gold). In this event, the incident light photons are absorbed, and the energy is transferred to the electrons, which convert into surface plasmons (collective electron oscillations). The binding of biomolecules on the nanostructure metal surface results in reduction of energy intensity of the reflected light, so-called a spectral shift (i.e., the reflected light has lower energy intensity and a longer wavelength compared to the incident light). The spectral shift, which can be determined in real-time, is linear and extremely sensitive to the number of molecules bound to the nanostructure surface of sensors. The superb sensitivity of LSPR spectral shift makes it possible to measure the binding of molecules on the nanostructure metal surface and their eventual interactions with specific ligands accurately. Utilizing unique advantages of nanostructure-based LSPR, including free-label sensing ability, exquisite sensitivity, precise, real-time measuring, and instrument simplicity, the present disclosure describes a wearable biosensor device for detecting SARS-CoV-2 antigens in body fluids.

In recent years, the development of nanofabrication enables nanoparticles to be fabricated for LSRP sensing platforms. Nanoparticle-based LSRP has been successfully applied to detect biomarkers of Alzheimer's disease in human cerebrospinal fluid samples. However, several challenges remain, which limit the use of metal nanoparticles in many biosensor applications. Improving the limit of detection (LOD) of nanoparticles-based LSRP is problematic since the detection of small molecules requires a large amount of biomolecular material to coat the surface. As of Apr. 23, 2020, Jing Wang and his team at the Swiss Federal Laboratories for Materials Science and Technology (Empa) and ETH Zurich (Swiss Federal Institute of Technology in Zurich) have been working to develop an LSRP-based biosensor for detecting SARS-CoV-2 using a DNA receptor sequence that is complimentary to the SARS-CoV-2 RNA genome. These DNA stands are bound on the gold nanoparticle glass substrate. Since viral RNAs are small molecules, it is not clear how the LOD of the biosensor can be improved. At the early phase of infection, when viral material represents at low levels in human body fluid, the low LOD—or low sensitivity of diagnostic assays can miss out asymptotic carries.

Moreover, non-specific binding between RNA-DNA can occur at the ambient temperature and can lead to a false positive. To overcome this problem, the Wang team suggests heating the nanostructures with a laser of a specific wavelength to create the correct heat to ensure that only fully complementary strands bind. However, integrating a laser generator and a thermal control unit into LSRP biosensors can be a significant obstacle to the miniaturization process. Therefore, no wearable devices for reliably detecting SARS-CoV-2 have been successfully developed.

To this end, the present disclosure presents a wearable device for detecting SARS-CoV-2 antigens in body fluids using nanostructure-based Localized Surface Plasmon Resonance (LSPR). There are several advantages to an exemplary nanostructured-based LSPR biosensor device. Firstly, the device is designed to detect SARS-CoV-2 nucleocapsid proteins (N proteins)—the most abundant viral structural protein. Of note, there are hundreds of N proteins binding one viral genome, and the viral RNA: N protein molar ratio is from 1:17 to 1:71. This feature significantly enhances the sensitivity of detection. Also, most of the SARS-CoV-1 patients had the detectable N protein in a variety of body fluids (including serum, nasal aspirates, urine, and stool samples) at the early stage of the disease. Due to the highly similar viral structure (especially N protein) between SARS-CoV-1 and SARS-CoV-2, N proteins are more likely a good candidate for a rapid and sensitive diagnostic strategy for SARS-CoV-2. Secondly, an exemplary nanostructured-based LSPR biosensor device detects the binding of N proteins and their validated specific antibodies (protein-protein interaction). Such protein-protein interaction is not suffered from the non-specific binding occurring at the ambient temperature. Therefore, there are no needs for a thermal control unit to ensure accuracy. Such instrument simplicity makes miniaturization possible. Thirdly, unlike all single-use testing devices currently available on the market, an exemplary nanostructured-based LSPR biosensor device is designed to allow end-users to perform the testing multiple times. Taking advantage of the electric-modulated reversible binding between antigens and antibodies, an exemplary biosensor device can be designed to use electric current from smartphones to dissociate non-covalent binding between antigens-antibodies. Of note, many SARS-CoV-2 carriers are undetected at the testing time due to the low viral load. Therefore, multiple tests need to be performed at different timepoints as the infection progresses to ensure not excluding carrier individuals. Although it is a time consuming and expensive strategy, many federal governments have implemented this strategy to prevent the spread of the disease. Accordingly, an exemplary nanostructured-based LSPR biosensor device will help to ease the COVID-19 burden on communities and healthcare systems.

Since there are no specific treatments or vaccines available for SARS-CoV-2 (as of 2020), social distancing and community testing are only effective measures to prevent the spread of the disease. However, community testing requires a mass workload for public health sectors and advanced biosafety level 3 laboratories, which is not practical for large-scale testing campaigns. Therefore, there are an urgent need and enormous demands for a rapid, reliable, simple, portable, and inexpensive COVID-19 testing device that can help to decentralize COVID-19 testing and make community testing feasible. Since a nanostructured-based LSPR biosensor device can be built on smartphone platforms, it can be used as an at-home test for COVID-19, in accordance with various embodiments of the present disclosure. Potential markets and customers will be central labs, universities, hospitals, residential households, schools, etc.

Correspondingly, the present disclosure describes certain systems and methods for employing nanostructure-based LSPR to detect N proteins of SARS-CoV-2. In various embodiments, a biosensor device comprising the nanostructure-based LSPR biosensor can be interfaced with a mobile device, such as a smartphone, in which a smartphone LED flash or flashlight can be used as a light source for illuminating the biosensor device and a smartphone camera can be used as a spectrometer for LSRP readout. Thus, in various embodiments, a biosensor testing application on a smartphone (or other computing device) can detect changes in spectral shift of incoming video or static images indicating a presence of a particular biomaterial on the biosensor. Additionally, smartphone electric current (e.g., an exemplary biosensor can be coupled to a phone port, such as a microUSB port, as an example) can be used to dissociate the antigen-antibody binding, in some embodiments, such that the nanostructured-based LSPR biosensor can be reset for multiple tests. Accordingly, a novel nanostructured-based LSPR biosensor device of the present disclosure is capable of detecting SARS-CoV-2 antigens (virus particles) in body fluids using nanostructure-based localized surface plasmon resonance (LSPR).

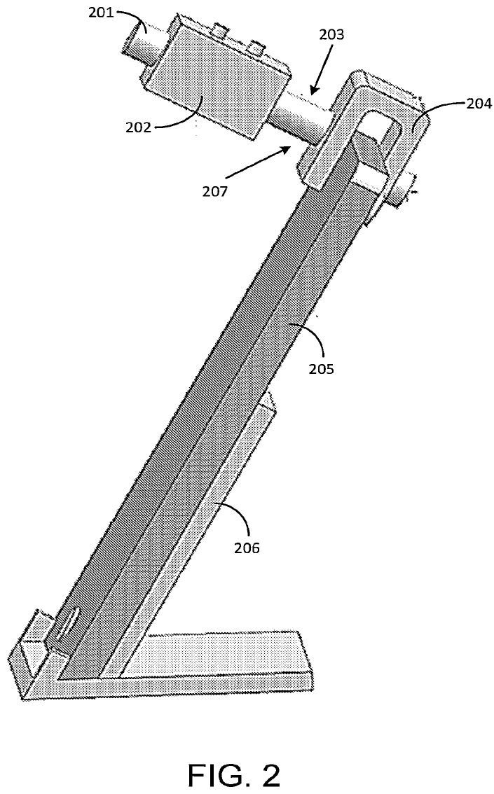

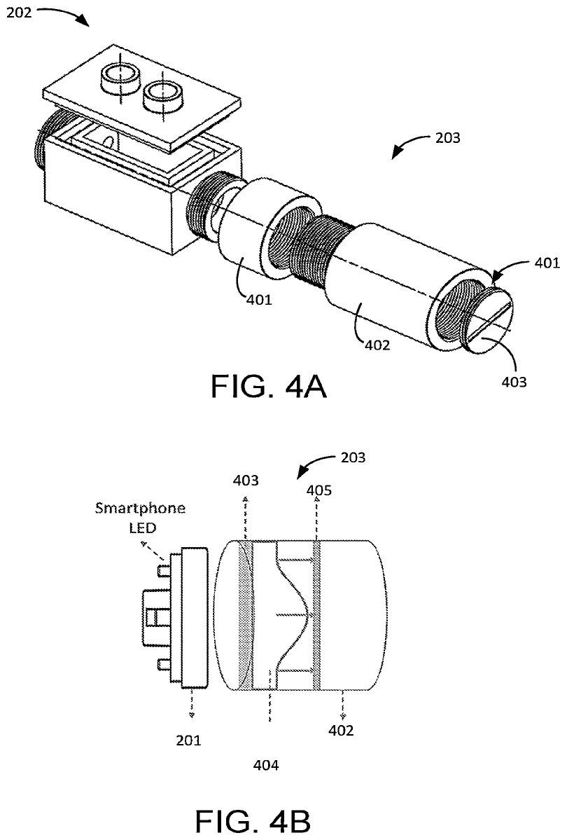

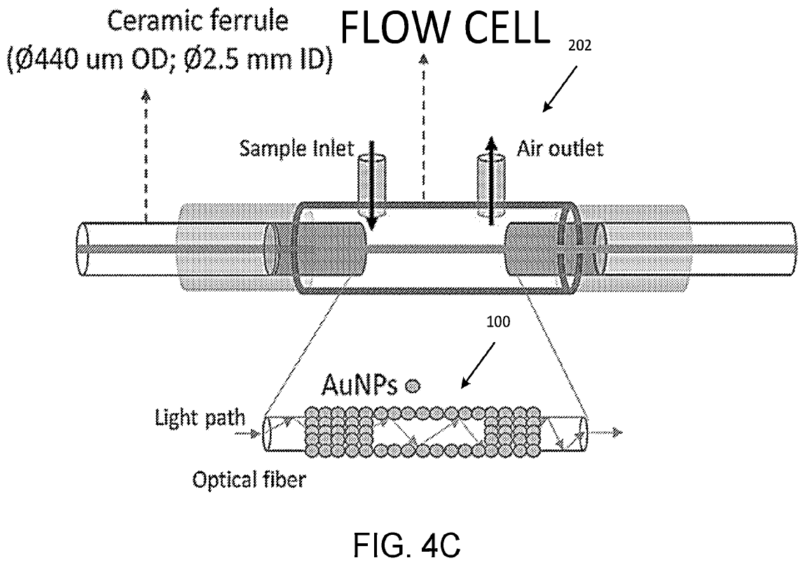

As discussed, an exemplary biosensor device comprising the nanostructure-based LSPR can be interfaced with a mobile device. Correspondingly, FIG. 2 illustrates an exemplary smartphone-based biosensor system in accordance with various embodiments. Such a system includes a flow cell 202 with a LED generator 201 at one end and an optical tube 203 at another end. The optical tube 203 is mounted on a clip 204 (e.g., plastic clip) placed directly on top (e.g., as close as possible) of camera lens or sensor 207 (e.g., the smartphone camera lens). The smartphone 205 can be supported by a smartphone stand 206. In various embodiments, the flow cell 202 is connected to the LED generator 201 and/or the optical tube 203 by threaded connection(s). In various embodiments, different arrangements are contemplated where the smartphone 205 can be used as the LED generator 201 via the smartphone's LED source, as discussed below in connection with FIG. 4A. In such a scenario, an optical fiber spectrometer can be used to measure an intensity or spectrum of the output light path from the flow cell (whether it be absorbed, reflected, transmitted, or scattered light).

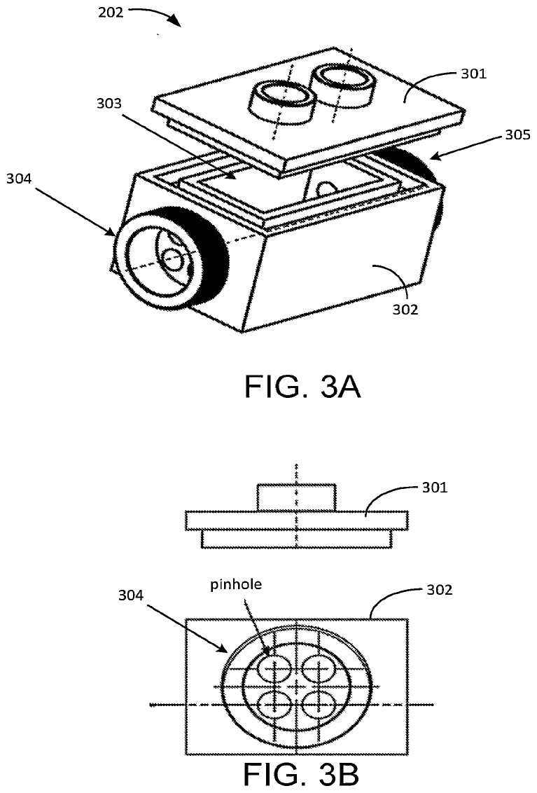

FIGS. 3A and 3B illustrates varying views of a detailed drawing of an embodiment of the flow cell 202. The flow cell 202 includes two key components: a top portion 301 and a base portion 302. The top portion 301 has inlet and outlet holes, in which the inlet and outlet holes have same the inside and outside diameter (ID and OD) and can be used interchangeably. In various embodiments, among others, the top portion 301 sits on and engages the base portion 302 by a scarf joint running along a top surface of the base portion 302. In various embodiments, the base portion 302 has, but is not limited to only having, three components: a chamber 303 and two male threaded connectors 304, 305 at two ends, in which the two male connectors have the same ID, OD, and thread profile and can be used interchangeably. In various embodiments, the chamber 303 has 8 identical pinholes (4 holes on each end, as demonstrated by FIG. 3B), where the pinholes on one end are concentric with the pinholes on the other end. In various embodiments, each pinhole pair houses an exemplary nanostructure-based LSPR biosensor 100 in the form of a nanostructured-based fiber optic (FO) LSPR biosensor, in accordance with various embodiments of the present disclosure. Thus, an exemplary chamber 303 houses four FO-LSPR biosensors (one biosensor sensing SARS-CoV-2 N protein for the virus test, one biosensor sensing SARS-CoV-2 antibodies against SARS-CoV-2 S protein for the antibody test, one biosensor used as isotype controls for the specificity, and one biosensor used as a reference control to normalize the fluctuation of smartphone LED intensity).

Next, FIG. 4A illustrates an embodiment of the optical tube 203, in which the optical tube includes one female-threaded connector 401, a threaded-fitting adapter 402, and an optical component, such as an external-threaded entrance slit 403. In various embodiments, the threaded-fitting adapter 402 accommodates the optical component (e.g., the entrance slit 403) via a female-threaded end, and the female-threated connector 401 joints the male-threaded end of a flow cell 202 with a male-threaded end of the optical component.

In various embodiments, as shown in FIG. 4B, the optical tube 203 includes an external-threaded entrance slit 403, aspheric condenser lens 404 (e.g., having dimensions of Ø10 mm, f=8 mm, NA=0.61), and a colored glass filter 405 (e.g., having dimensions of 1.2 mm thickness and 420 nm long pass). Accordingly, an end of the optical component having the entrance slit 403 can be positioned next to an LED generator 201, whether it be that of a smartphone LED or an external LED generator.

In accordance with the present disclosure, an exemplary chamber of 303 of an exemplary flow cell 202 holds or contains nanostructured-based fiber optic (FO)-LSPR biosensor(s) 100. In an exemplary assembly process, two ends of a nanostructured-based FO-LSPR biosensor 100 is secured by ceramic ferrules and connected to the chamber 303 via concentric holes on two ends of the chamber such that the biosensor extends across a length of the chamber 303 from end to end and is coupled to respective pinholes at each end of the chamber. Accordingly, one end of the flow cell 202 can be connected to a LED generator 201 via an LED adapter, and the other end of the flow cell can be coupled to an optical tube 203 that is aligned with a lens or sensor of a camera 207 of a smartphone. In various embodiments, the smartphone is configured to capture image(s) of exiting light from the pinholes of the flow cell chamber 303 and transform the captured image(s) into grayscale and calculate an average intensity of the images for each pinhole/biosensor deployed in the flow cell 202. Accordingly, in various embodiments, a biosensor testing application stored and executed on the smartphone can detect changes in a spectral shift or light intensity of incoming video or single images. As such, the biosensor testing application contains computer instructions for performing the applicable image acquisition and sensor data analysis routines in accordance with the disclosed methods of the present disclosure.

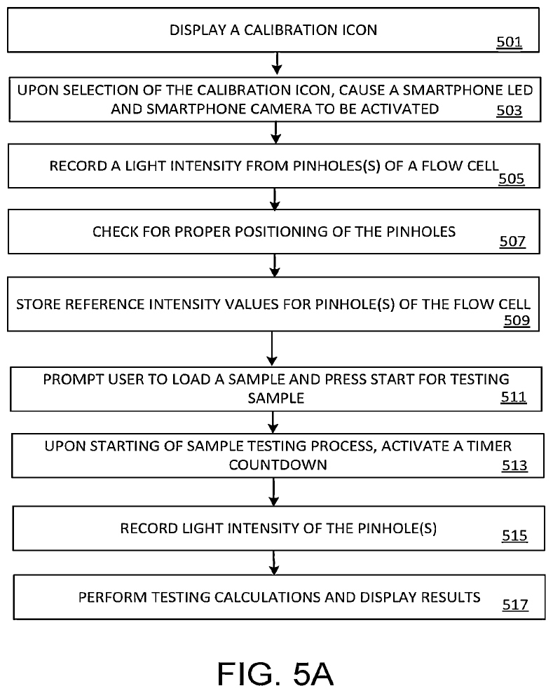

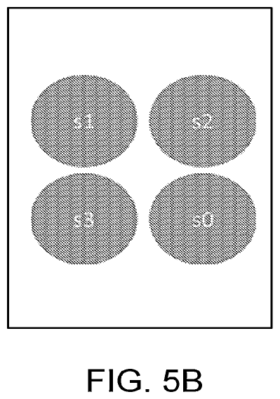

Referring now to FIG. 5A, an exemplary flow diagram of an exemplary biosensor testing application is provided, such as that which may be executed by a smart phone. Under step 501, a “Calibration” icon is displayed by the application (e.g. on a smartphone graphical user interface) that can be selected or “clicked” by a user. Selection of the calibration icon will cause a smartphone LED and smartphone front camera to be activated and after a period of time (e.g., 30 seconds), a light intensity from the respective pinholes of a flow cell to be recorded, as shown in step 503. Correspondingly, FIG. 5B shows a layout of the respective pinholes of the flow cell which can be displayed by the biosensor testing application. Thus, in step 505, the biosensor testing application records the light intensity from Spot S1, S2, S3, and S0, where the 51 biosensor senses SARS-CoV-2 N protein for the virus test, the S2 biosensor senses SARS-CoV-2 antibodies against SARS-CoV-2 S protein for the antibody test, the S3 biosensor is used as isotype controls for the specificity, and the S0 biosensor is used as a reference control to normalize the fluctuation of smartphone LED intensity. Correspondingly, a proper positioning of the pinhole(s) is checked in step 507. For example, if

$$\frac{\vert {s}_{i} - {s}_{0} \vert}{{s}_{0}} \geq 0.2 ( i = 1 , 2 , 3 ) ,$$

which indicates that the pinholes may not be properly aligned with the camera, the biosensor testing application can display a message advising to “Please adjust the pinhole in the middle of the smartphone front camera.” Else, in step 509, if the pinhole(s) are properly positioned, an exemplary biosensor testing application stores the reference intensity values for respective pinholes S1, S2, S3, S0 as S1₀, S2₀, S3₀, S0₀. Next, in step 511, the biosensor testing application displays a prompt to “Please load your sample and press start (icon)” which, when pressed, causes the smartphone LED and camera to be deactivated before initiation of testing of the loaded sample, in step 511. Upon clicking of the “Start” icon, a timer countdowns (e.g., 10 minutes) is activated and displayed by the biosensor testing application, in step 513. At completion of the timer countdown, the smartphone LED and smartphone camera is activated by biosensor testing application and a new timer countdown (e.g., 30 seconds) is initiated. Then, in step 515, after completion of the timer countdown, a light intensity of the pinhole(s) (corresponding to displayed pinholes S1, S2, S3 and S0) is recorded and stored as new values S1₁, S2₁, S3₁, S0₁. Next, in step 517, calculations are performed for the recorded intensity values and results are displayed. Accordingly, if

$$\frac{s {1}_{0} - s {1}_{1}}{s {0}_{0} - s {0}_{1}} < 1 ,$$

then the biosensor testing application displays an “S1 is positive” message; else, the biosensor testing application displays an “S1 is negative” message. Correspondingly, if

$$\frac{s {2}_{0} - s {2}_{1}}{s {0}_{0} - s {0}_{1}} < 1 ,$$

then the biosensor testing application displays an “S2 is positive” message. Additionally, if

$$\frac{s {3}_{0} - s {3}_{1}}{s {0}_{0} - s {0}_{1}} < 1 ,$$

the biosensor testing application displays an “S3 is positive” message; else, the biosensor testing application displays an “S3 is negative” message.

The present disclosure includes an exemplary process for fabricating a nanostructured-based fiber-optic LSPR (FO-LSPR) biosensor that is capable of detecting the presence of SARS-CoV-2 N protein. Accordingly, an initial step involves the cleaning and hydroxylation process of optical fiber. First, the most-outer hard plastic layer of optical fiber (Ø400 μm Core TECS-Clad Multimode Optical Fiber, 0.39 NA) (OF) can be removed using concentrated Acetone in 30 min. Then, the hydroxylation of OF can be accomplished by immersing the OF for 40 min in a mixed solution of concentrated H₂SO₄ and H₂O₂. Subsequent steps are related to sensing film preparation. Accordingly, a polyelectrolyte multiplayer (PE) self-assembly method may be utilized to prepare gold nanoparticles (NP) sensing film, in which the OF can be alternately immersed in three kinds of PE solutions: 0.5% of Poly-diallyl dimethylammonium chloride (PDDA), 1 mg/ml of Poly(sodium 4-styrene sulfonate) (PSS) and 1 mg/ml of poly(allylamine hydrochloride) (PAH). The assembly time for each assembly process is 15 min. Then, the OF 15 min immersed in 40 nm AuNPs in 60 min to generate AuNP_OF. Two ends of the AuNP-coated optical fibers may then be put through a ceramic ferrule (Ø440 um OD; Ø2.5 mm ID) and cured with epoxy. The cured optical fiber-ferrules may then be polished using polishing papers in five steps. The first four steps using diamond sheets in four different grit sizes of 30, 6, 3, and 1 μm, and the fifth using our final polishing sheets which have a grit size of 0.02 μm. Then, OF can be washed five times with PBS (Phosphate Based Saline) (200 μl each time), and the OFs can be incubated with 200 μl of 5 μg/ml SARS-CoV-2 antibodies (specific for SARS-CoV-2 N protein) for 1 hour at room temperature, following by 5 times washing with PBS (200 μl each time). After the washing, the OFs may be incubated with 5% bovine serum albumin in PBS for 1 hour at room temperature. The immune-modified OFs are now ready to use.

Referring now to FIG. 4C, a schematic of an exemplary nanostructured-based LSPR biosensor is shown within a flow cell 202 in accordance with embodiments of the present disclosure. In particular, the top of the figure shows the nanostructured-based LSPR biosensor 100 positioned within the flow cell 202, where the bottom of the figure shows an enlarged view of a portion of the nanostructured-based LSPR biosensor 100. Accordingly, the enlarged view shows the nanostructured-based LSPR biosensor 100 in the form of an AuNP-coated optical fiber and demonstrates the path of light as it traverses a length of the AuNP-coated optical fiber and comes in contact with the metal surface (AuNPs) of the nanostructured-based LSPR biosensor 100.

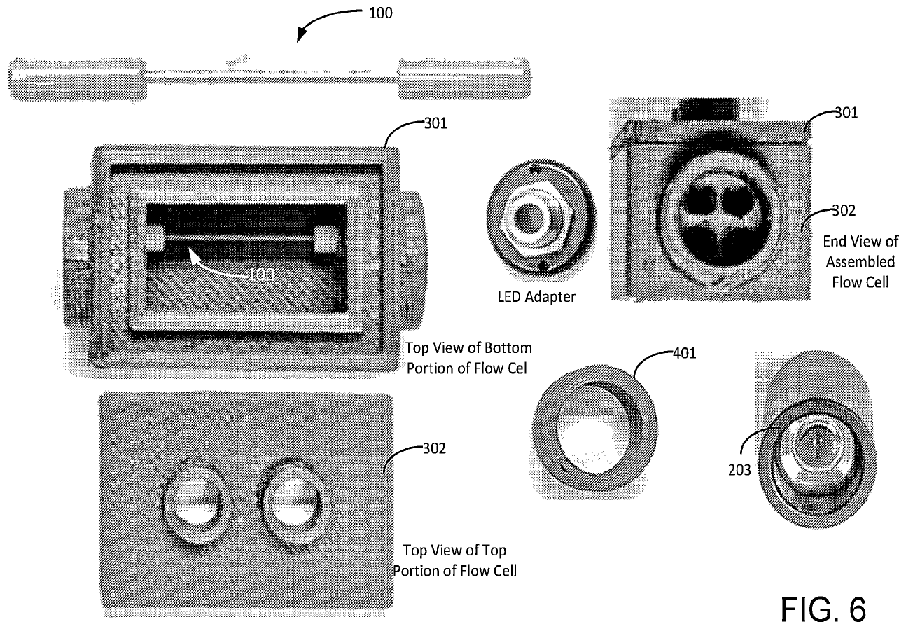

Correspondingly, FIG. 6 shows a composite of images of prototype components of an exemplary flow cell assembly 202 in accordance with embodiments of the present disclosure. The image shows a prototype nanostructured-based fiber optic LSPR biosensor 100 having ceramic ferrules, and varying views of an exemplary flow cell 202 is shown, including a view with an exemplary nanostructured-based FO LSPR biosensor 100 positioned across a length of a chamber 303 of the flow cell 202. Further, additional connectors are also provided including an LED adapter 402, female threaded connector 401, and an optical tube 203 with an optical lens and glass filter.

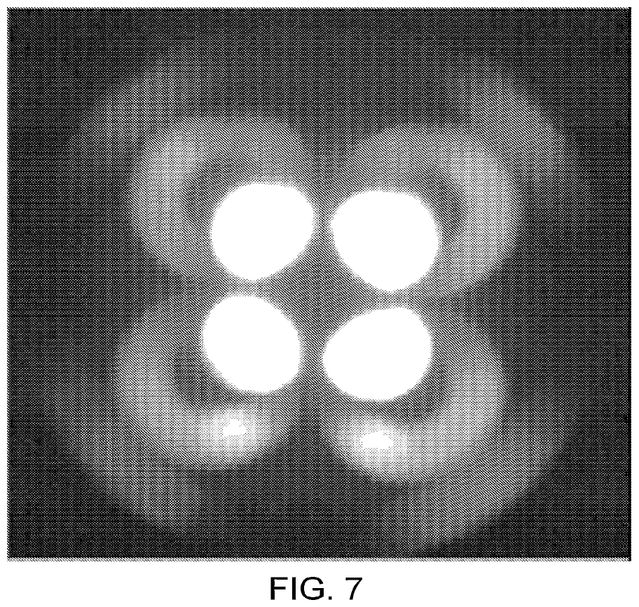

In an exemplary implementation, among others, of the exemplary nanostructured-based LSPR biosensor 100 and flow cell assembly, 200 μl of a sample (e.g., respiratory fluids, serum, plasma or cell culture supernatants) can be collected in 1.5 ml tube and diluted with 1 ml PBS. 1 ml of the diluted sample can then be aspirated using a 1 ml syringe. After the aspiration, the syringe can be connected to 0.22 um syringe-filter unit. The sample can then be flushed or injected into the flow cell 202 via the inlet hole on a top portion 301 of the flow cell 202, in which the outlet hole on the top portion 301 of the flow cell 202 allows air within the chamber 303 to exit. The sample within the flow cell 202 and nanostructured-based LSPR biosensor 100 may be incubated at room temperature for at least 10 min. After the incubation, image(s) are captured or acquired by a camera sensor positioned to acquire image(s) of the exiting light from the pinholes of the flow cell chamber 303, as represented in FIG. 7. In certain embodiments, a camera sensor of a smartphone 205 can be used to acquire the aforementioned images such that the smartphone 205 is configured to calculate an average intensity for each of the pinholes, where the presence of a particular biomaterial is indicated by a change in intensity.

One exemplary method, in accordance with various embodiments of the present disclosure, includes the operations of providing a nanostructure-based LSPR (localized surface plasmon resonance) biosensor; illuminating a metal surface of the nanostructure-based LSPR biosensor with a monochromatic, broadband or laser light; measuring a reference intensity of absorbed, reflected, transmitted or scattered light from the nanostructure-based LSPR biosensor; applying a linker molecule or polymer chain or nucleic acid (DNA, RNA) or peptide strand or capture antibody or polydopamine or alkane thiol or synthetic molecules with varying carbon chains comprised of —NH₂ or —COOH or —SH group at one end or both ends of the molecules to the metal nanostructure LSPR biosensor; applying intermediate layer comprised of polyethylene glycol (PEG) or streptavidin or avidin or biotin or Polyethylenimine (PEI) or polyaziridine or dextran to the linker molecule; applying probe antibodies or primary antibodies and/or secondary antibodies to the intermediate layer; applying metal nanostructure LSPR biosensor to paper-based microfluidics platforms or silicon-based microfluidic chips applying a body fluid sample to the metal surface of the nanostructure-based LSPR biosensor with linker, intermediate, and capture/probe antibodies; illuminating the metal surface of the nanostructure-based LSPR biosensor with the light comprising of monochromatic or broadband or laser; measuring an intensity of absorbed, reflected, transmitted or scattered light from the nanostructure-based LSPR biosensor having the body fluid sample and comparing the measured intensity with the reference intensity; detecting a spectral shift of the absorbed, reflected, transmitted or scattered light from the nanostructure-based LSPR biosensor having the body fluid sample; and/or signaling that the body fluid sample is positive for a presence of a particular biomaterial in response to detecting the spectral shift of the absorbed, reflected, transmitted or scattered light, wherein the biomaterial has binded or adsorbed to the nanostructure metal surface.

In various embodiments, the biomaterial comprises S, N, or M proteins of SARS-CoV-2; a light source for the light is a smartphone LED flash, standalone LED, laser, continuum laser light, pulsed laser light, or broadband source; and/or an internal camera 207 of the smartphone or charged coupled device (CCD) or photometer or photodiode is used in measuring the intensity of the absorbed, reflected, transmitted or scattered light from the nanostructure-based LSPR biosensor.

In various embodiments, operations of such a method may further include applying an electric current to the nanostructure-based LSPR biosensor to disassociate any binding of the biomaterial with surface of the nanostructure-based LSPR biosensor, wherein the electric current may be supplied by the smartphone or external battery or power source; and/or reusing the nanostructure-based LSPR biosensor to test for the presence of the biomaterial in a new body fluid sample.

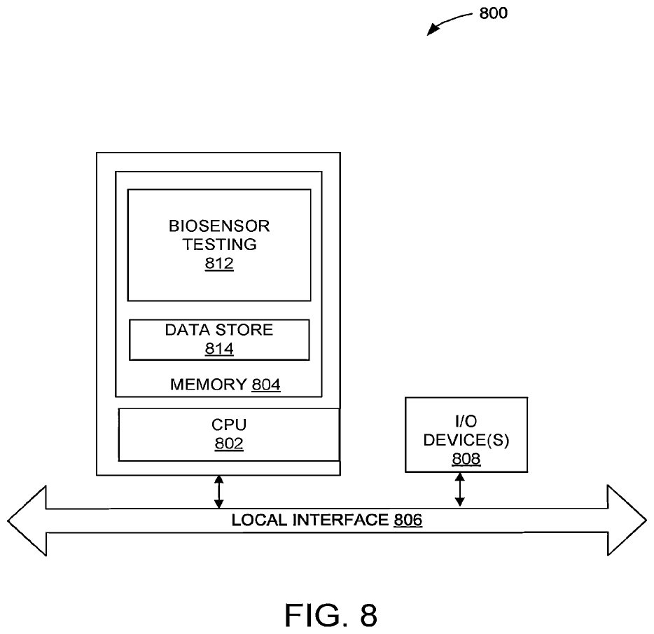

Referring now to FIG. 8, the figure provides a schematic of a computing device 800 in accordance with various embodiments of the present disclosure. In example embodiments, the computing device 800 may be, but is not limited to, a smartphone device 205 that is equipped with an LED light source (such as LED flash or flashlight) and a camera 207. An exemplary computing device 800 includes at least one processor circuit, for example, having a processor (CPU) 802 and a memory 804, both of which are coupled to a local interface 806, and one or more input and output (I/O) devices 808. The local interface 806 may comprise, for example, a data bus with an accompanying address/control bus or other bus structure as can be appreciated.

Stored in the memory 804 are both data and several components that are executable by the processor 802. In particular, stored in the memory 804 and executable by the processor 802 may be software, such as an exemplary biosensor testing application 812 that is capable of determining a presence of a particular biomaterial on an exemplary nanostructure LSPR biosensor of the present disclosure, and potentially other related data. In addition, an operating system may be stored in the memory 804 and executable by the processor 802. The I/O devices 808 may include input devices, for example but not limited to, a keyboard, video cameras, etc. Furthermore, the I/O devices 808 may also include output devices, for example but not limited to, display, etc. Also, the I/O devices 808 may include a communication component, such as a network adapter or interface (e.g., WiFi network adapter, Bluetooth adapter, 4G wireless adapter, ethernet adapter, etc.), that allows for wired or wireless communications with external devices and networks.

Certain embodiments of the present disclosure can be implemented in hardware, software, firmware, or a combination thereof. If implemented in software, exemplary biosensor testing logic or functionality are implemented in software or firmware that is stored in a memory and that is executed by a suitable instruction execution system. If implemented in hardware, the biosensor testing logic or functionality can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

It should be emphasized that the above-described embodiments of the present disclosure are merely possible examples of implementations, merely set forth for a clear understanding of the principles of the disclosure. Many variations and modifications may be made to the above-described embodiment(s) without departing substantially from the principles of the disclosure. All such modifications and variations are intended to be included herein within the scope of this disclosure and protected by the following claims.

1. A method comprising:
illuminating a metal surface of a nanostructure-based LSPR (localized surface plasmon resonance) biosensor directly or using an optical fiber with a monochromatic, broadband, or laser light;
measuring a reference intensity of absorbed, reflected, transmitted or scattered light from the nanostructure-based LSPR biosensor;
applying a linker molecule or polymer chain or nucleic acid (DNA, RNA) or peptide strand or capture antibody or polydopamine or alkane thiol or synthetic molecules with varying carbon chains comprised of —NH₂ or —COOH or —SH group at one end or both ends of the molecules to the metal nanostructure LSPR biosensor;
applying an intermediate layer comprised of polyethylene glycol (PEG) or streptavidin or avidin or biotin or Polyethylenimine (PEI) or polyaziridine or dextran to the linker molecule;
applying probe antibodies or primary antibodies and/or secondary antibodies to the intermediate layer;
applying a body fluid sample comprised of saliva, blood, sweat, tear, or cerebrospinal fluid to a metal surface of the nanostructure-based LSPR biosensor with linker, intermediate, and capture/probe antibodies;
illuminating the metal surface of the nanostructure-based LSPR biosensor with the monochromatic, broadband, or laser light;
measuring an intensity of absorbed, reflected, transmitted, or scattered light from the nanostructure-based LSPR biosensor having the body fluid sample and comparing the measured intensity with the reference intensity;
detecting a spectral shift of the absorbed, reflected, transmitted, or scattered light from the nanostructure-based LSPR biosensor having the body fluid sample; and
signaling that the body fluid sample is positive for a presence of a particular biomaterial in response to detecting the spectral shift of the absorbed, reflected, transmitted, or scattered light, wherein the biomaterial has binded or adsorbed to the metal surface of the nanostructure-based LSPR biosensor.

2. The method of claim 1, wherein the biomaterial comprises S, N, or M proteins of SARS-CoV-2.

3. The method of claim 1, wherein a light source for the light is a smartphone LED flash, standalone LED, laser, continuum laser light, pulsed laser light, or broadband source, wherein a delivery of light utilizes an optical fiber.

4. The method of claim 3, wherein an internal camera of the smartphone or charged coupled device (CCD) or photometer or photodiode is used in measuring the intensity or spectrum of the absorbed, reflected, transmitted or scattered light from the nanostructure-based LSPR biosensor, wherein a collection of light is made via the optical fiber.

5. The method of claim 4, further comprising applying an electric current to the nanostructure-based LSPR biosensor to disassociate any binding of the biomaterial with surface of the nanostructure-based LSPR biosensor, wherein the electric current is supplied by the smartphone or external battery or power source.

6. The method of claim 1, further comprising applying an electric current to the nanostructure-based LSPR biosensor to disassociate any binding or adsorption of the biomaterial with surface of the nanostructure-based LSPR biosensor.

7. The method of claim 6, further comprising reusing the nanostructure-based LSPR biosensor to test for the presence of the biomaterial in a new body fluid sample.

8. The method of claim 1, wherein the nanostructure-based LSPR biosensor comprises an Au (gold) or Ag (silver) or Al (aluminum) or Cu (copper) or alloy of one of these nanoparticles (NP) coated fiber optic LSPR biosensor, wherein a shape of the nanoparticles is a sphere, cube, triangle, star, or rod shape, wherein the NP can be coated with oxide materials made of SiO₂, TiO₂, MnO₂ or other metal oxides to make a core-shell structure.

9. The method of claim 1, further comprising:
inserting the nanostructure-based LSPR biosensor in a chamber of a flow cell, wherein the body fluid sample is applied to the nanostructure-based LSPR biosensor by being injected into an inlet hole of the flow cell.

10. The method of claim 9, wherein the metal surface of the nanostructure-based LSPR biosensor is illuminated by passing the monochromatic, broadband, or laser light into a hole at one end of the flow cell that is coupled to one end of the nanostructure-based LSPR biosensor.

11. The method of claim 10, further comprising capturing an image of the monochromatic, broadband, or laser light exiting from a hole at an opposite end of the flow cell that is coupled to an opposite end of the nanostructure-based LSPR biosensor, wherein the intensity of the absorbed, reflected, transmitted or scattered light from the nanostructure-based LSPR biosensor having the body fluid sample is determined by computing an average intensity of the captured image.

12. The method of claim 9, wherein each end of the flow cell contains a plurality of holes for holding a plurality of nanostructure-based LSPR biosensors, the plurality of nanostructure-based LSPR biosensors including a biosensor for sensing SARS-CoV-2 N protein, a biosensor for sensing SARS-CoV-2 antibodies against SARS-CoV-2 S protein, a biosensor used as isotype controls for specificity, and a biosensor used as a reference control.

13. A system comprising:
a nanostructure-based LSPR (localized surface plasmon resonance) biosensor having a linker molecule or polymer chain or nucleic acid (DNA, RNA) or peptide strand or capture antibody or polydopamine or alkane thiol or synthetic molecules with varying carbon chains comprised of —NH₂ or —COOH or —SH group at one end or both ends of the molecules to the nanostructure-based LSPR biosensor; an intermediate layer comprised of polyethylene glycol (PEG) or streptavidin or avidin or biotin or Polyethylenimine (PEI) or polyaziridine or dextran to the linker molecule; and probe antibodies or primary antibodies and/or secondary antibodies applied to the intermediate layer, wherein opposing ends of the biosensor are secured with ceramic ferrules; and
a flow cell having an internal chamber, a top portion, a bottom portion, and opposing end portions, wherein the nanostructure-based LSPR biosensor is insertable within the internal chamber and is held in place by engaging the ends of the biosensor within holes at each end of the internal chamber;
wherein the holes at each end of the internal chamber are configured to pass incoming light from one end of the flow cell to the opposite end of the flow cell across a length of the nanostructure-based LSPR biosensor;
wherein a top portion of the flow cell includes an inlet hole for inserting a body fluid sample to the nanostructure-based LSPR biosensor.

14. The system of claim 13, further comprising:
a light source positioned at one end of the flow cell to provide the incoming light through the hole at the one end of the flow cell; and
a camera positioned at an opposite end of the flow cell to capture an image of exiting light from hole at the opposite end of the flow cell.

15. The system of claim 13, wherein the nanostructure-based LSPR biosensor comprise an Au/Ag/Al/Cu/Fe/Mn, or their alloy, or their oxide NP-coated fiber optic LSPR biosensor.
